(19) United States
(12) Patent Application Publication
(10) Pub. No.: US 2026/0049962 A1
(43) Pub. Date: Feb. 19, 2026

(54) CONDITION DETERMINING APPARATUS, METHOD, AND RECORDING MEDIUM

(71) Applicants: ADVANTEST CORPORATION, Tokyo (JP); National University Corporation, Iwate University, Iwate (JP)

(72) Inventors: Yuji OGATA, Miyagi (JP); Tomonori YANAGIDA, Miyagi (JP); Koichiro KOBAYASHI, Iwate (JP)

(73) Assignees: ADVANTEST CORPORATION, Tokyo (JP); National University Corporation, Iwate University, Iwate (JP)

(21) Appl. No.: 19/191,066

(22) Filed: Apr. 28, 2025

(30) Foreign Application Priority Data
Aug. 15, 2024 (JP) 2024-135723

(51) Int. Cl. G01N 27/90; G01N 33/20

(52) U.S. Cl. CPC G01N 27/9073; G01N 33/20

(57) ABSTRACT

A condition determining apparatus includes an excitation section, a plurality of magnetic measuring sections, a correspondence recording section, and a condition determining section. The excitation section excites a measuring target inside an object. The magnetic measuring sections measure a magnetic field generated by an eddy current that is generated on the measuring target. The correspondence recording section records a correspondence between a condition of the measuring target and data based on measurement results from the magnetic measuring sections. The condition determining section determines the condition of the measuring target based on measurement results from the magnetic measuring sections and recorded contents in the correspondence recording section. The recorded contents in the correspondence recording section are recorded when the measuring target is placed at a plurality of respective depths in the object.

### BACKGROUND OF THE INVENTION

### Field of the Invention

The present invention relates to determining the condition (e.g. the presence of corrosion) of a measuring target (e.g., rebar) inside an object (e.g. ferroconcrete).

### Description of the Related Art

There have conventionally been proposed various methods for non-destructive inspection of rebar corrosion within a ferroconcrete. The current mainstream may include electrochemical methods such as a self-potential method and a polarization resistance method. Other methods have also been proposed such as those that uses electromagnetic radar, ultrasonic wave, and excitation heating. However, these depend largely on the measurement environment (the change in the specific permittivity due to humidity and moisture content) and the condition inside the concrete (the presence of cracks and cavities), suffering from a problem that low-precision results can only be obtained.

On the other hand, a magnetic field-based measurement may allow the influence of only the rebar to be measured with no influence from the above-described measurement environment or the above-described condition inside the concrete. Magnetic field-based methods for detection of rebar corrosion are described in Japanese Patent Application Publication Nos. H06-138094, 2020-012851, 2007-292572, 2001-194341, 2019-015655, 2018-025434, and 2019-128161.

Japanese Patent Application Publication Nos. H06-138094, 2020-012851, 2007-292572, and 2001-194341 each describe a method that does not utilize generation of an eddy current by an excitation coil. Such a method may suffer from various problems. For example, the problems include that it is required to cause a current to flow through the rebar, which cannot be a completely non-destructive inspection (Japanese Patent Application Publication No. H06-138094), that different rebars show different states of magnetization (Japanese Patent Application Publication No. 2020-012851), that it is required to generate a high magnetic field to magnetize the rebar (Japanese Patent Application Publication No. 2007-292572), and that it is not clear whether or not corrosion of the rebar, if placed at a deeper position in the ferroconcrete, can be detected as well as that the measurement result varies significantly depending on the surrounding environment (Japanese Patent Application Publication No. 2001-194341).

Japanese Patent Application Publication Nos. 2019-015655, 2018-025434, and 2019-128161 each describe a method that utilizes generation of an eddy current by an excitation coil. Note here that a single detector coil is used to detect an eddy current in Japanese Patent Application Publication Nos. 2019-015655, 2018-025434, and 2019-128161. It is also noted that Japanese Patent Application Publication Nos. 2016-105046, 2016-114533, 2020-003289, 2020-012851, and 2010-048723 each describe a method of detecting the condition of a measuring target by utilizing generation of an eddy current in the measuring target.

### SUMMARY OF THE INVENTION

However, even in the case where generation of an eddy current in a measuring target may be utilized to detect the condition of the measuring target, the measurement result of the eddy current depends on the depth of the measuring target (e.g. rebar) inside the object (e.g. ferroconcrete) and it is therefore difficult to detect the condition of the measuring target.

It is hence an object of the present invention to determine the condition of a measuring target inside an object based on an eddy current that is generated by exciting the measuring target, in which a difference in the eddy current at a different depth of the measuring target in the object is accommodated.

According to the present invention, a condition determining apparatus includes: an excitation section arranged to excite a measuring target inside an object; a plurality of magnetic measuring sections arranged to measure a magnetic field generated by an eddy current that is generated on the measuring target; a correspondence recording section arranged to record a correspondence between a condition of the measuring target and data based on measurement results from the magnetic measuring sections; and a condition determining section arranged to determine the condition of the measuring target based on measurement results from the magnetic measuring sections and recorded contents in the correspondence recording section, wherein the recorded contents in the correspondence recording section are recorded when the measuring target is placed at a plurality of respective depths in the object.

According to the above configured condition determining apparatus, an excitation section is arranged to excite a measuring target inside an object. A plurality of magnetic measuring sections are arranged to measure a magnetic field generated by an eddy current that is generated on the measuring target. A correspondence recording section is arranged to record a correspondence between a condition of the measuring target and data based on measurement results from the magnetic measuring sections. A condition determining section is arranged to determine the condition of the measuring target based on measurement results from the magnetic measuring sections and recorded contents in the correspondence recording section. The recorded contents in the correspondence recording section are recorded when the measuring target is placed at a plurality of respective depths in the object.

According to the condition determining apparatus of the present invention, the correspondence recording section may be arranged to record a correspondence between the condition of the measuring target as well as the depth of the measuring target and the data, the condition determining section may be arranged to determine the condition of the measuring target based on the depth of the measuring target, which is known, and the data may include measurement results from the magnetic measuring sections.

According to the condition determining apparatus of the present invention, the data may be obtained through multivariate analysis of measurement results from the magnetic measuring sections.

According to the condition determining apparatus of the present invention, the correspondence recording section may be arranged to record a correspondence between the condition of the measuring target as well as the depth of the measuring target and the data, the condition determining section may be arranged to determine the condition of the measuring target and further to measure the depth of the measuring target, and the data may be obtained through multivariate analysis of measurement results from the magnetic measuring sections.

According to the condition determining apparatus of the present invention, the correspondence may be obtained through machine learning with the condition of the measuring target and the measurement results from the magnetic measuring sections as training data.

According to the condition determining apparatus of the present invention, the correspondence may be obtained through machine learning with the condition of the measuring target, the depth of the measuring target, and the measurement results from the magnetic measuring sections as training data.

According to the condition determining apparatus of the present invention, the correspondence may vary depending on the depth of the measuring target.

According to the condition determining apparatus of the present invention, the object may be a ferroconcrete, and the measuring target may be a rebar.

According to the condition determining apparatus of the present invention, the condition may be whether or not the rebar is corroded.

According to the condition determining apparatus of the present invention, the condition may be whether or not the rebar is broken.

According to the condition determining apparatus of the present invention, a position of the rebar may be measured based on the measurement results from the magnetic measuring sections.

According to the condition determining apparatus of the present invention, a diameter or radius of the rebar may be measured based on the measurement results from the magnetic measuring sections.

According to the condition determining apparatus of the present invention, the condition determining section may be arranged to determine the condition of the measuring target based on some of the measurement results from the magnetic measuring sections.

According to the present invention, a condition determining method includes: exciting a measuring target inside an object; measuring a magnetic field generated by an eddy current that is generated on the measuring target; recording a correspondence between a condition of the measuring target and data based on measurement results from the measuring; and determining the condition of the measuring target based on measurement results from the measuring and recorded contents in the recording, wherein the recorded contents in the recording are recorded when the measuring target is placed at a plurality of respective depths in the object.

The present invention is a non-transitory computer-readable medium including a program of instructions for execution by a computer to perform a condition determining process with using a condition determining apparatus having an excitation section arranged to excite a measuring target inside an object, and a plurality of magnetic measuring sections arranged to measure a magnetic field generated by an eddy current that is generated on the measuring target, the condition determining process including: recording a correspondence between a condition of the measuring target and data based on measurement results from the magnetic measuring sections; and determining the condition of the measuring target based on measurement results from the magnetic measuring sections and recorded contents in the recording, wherein the recorded contents in the recording are recorded when the measuring target is placed at a plurality of respective depths in the object.

### BRIEF DESCRIPTION OF THE DRAWINGS

FIG. 1 is a functional block diagram showing the configuration of a condition determining apparatus 1 according to a first embodiment of the present invention;

FIGS. 2 (a) and 2 (b) show a side view of a ferroconcrete (object) 2 (FIG. 2 (a)) and a b-b cross-sectional view (FIG. 2 (b));

FIGS. 3 (a) and 3 (b) show a front view in a state where a substrate 1s of the condition determining apparatus 1 is mounted to the ferroconcrete (object) 2 (FIG. 3 (a)) and a plan view of the substrate 1s of the condition determining apparatus 1 (FIG. 3 (b));

FIGS. 4 (a) and 4 (b) show an eddy current EC that is generated by the magnetic fields MF from the excitation section 1a in the cases where the normal rebar 2a is placed shallower (at a depth d1) (FIG. 4 (a)) and the normal rebar 2a is placed deeper (at a depth d2) (FIG. 4 (b));

FIG. 5 shows an eddy current EC that is generated by the magnetic fields MF from the excitation section 1a in the cases where the rebar 2a with corrosion 2c is placed shallower (at the depth d1); and

FIG. 6 illustrates determination of the condition of a measuring target (rebar 2a) by the condition determining section 1e according to the second embodiment.

### DESCRIPTION OF THE PREFERRED EMBODIMENT

Preferred embodiments of the present invention will hereinafter be described with reference to the accompanying drawings.

### First Embodiment

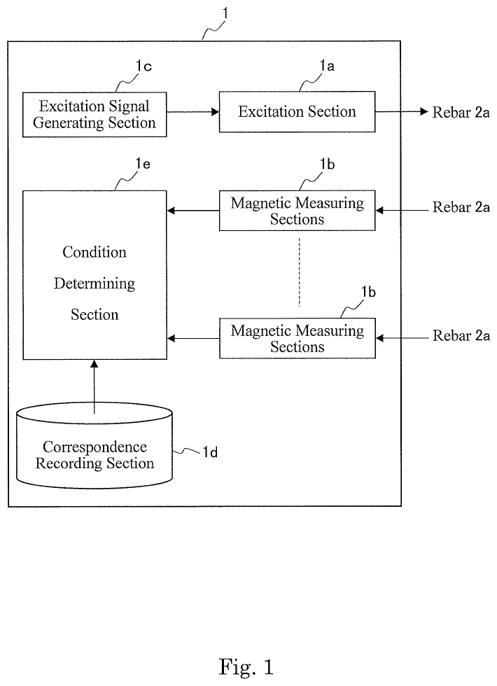

FIG. 1 is a functional block diagram showing the configuration of a condition determining apparatus 1 according to a first embodiment of the present invention. The condition determining apparatus 1 according to the first embodiment includes an excitation section 1a, magnetic measuring sections 1b, an excitation signal generating section 1c, a correspondence recording section 1d, and a condition determining section 1e.

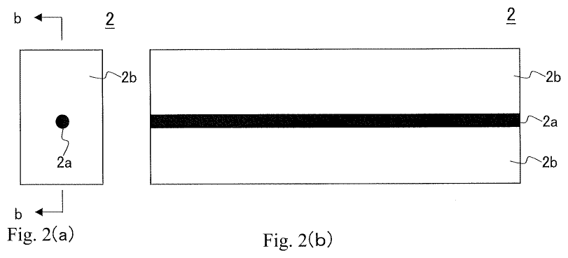

FIGS. 2 (a) and 2 (b) show a side view of a ferroconcrete (object) 2 (FIG. 2 (a)) and a b-b cross-sectional view (FIG. 2 (b)). Referring to FIGS. 2 (a) and 2 (b), the ferroconcrete 2 has a rebar 2a and a concrete 2b, the rebar 2a placed inside the ferroconcrete 2.

The excitation section 1a is arranged to excite a measuring target inside the object. As an example of the object and the measuring target, the embodiment of the present invention employs the ferroconcrete 2 as the object and the rebar 2a as the measuring target.

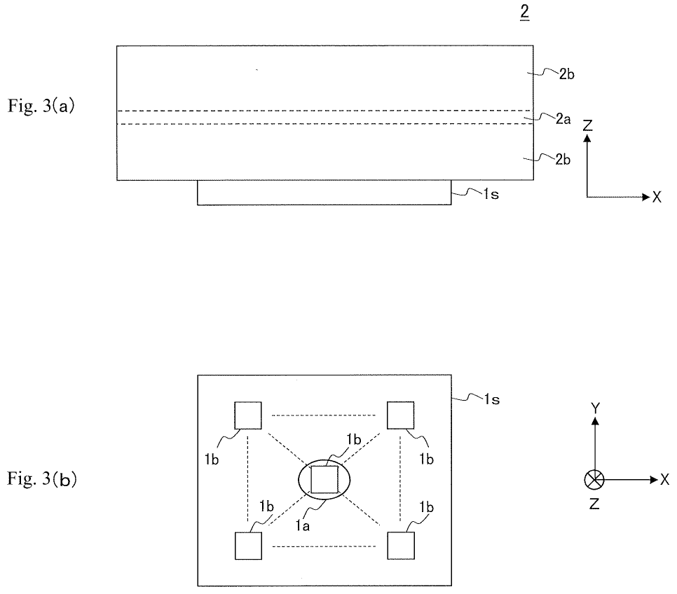

FIGS. 3 (a) and 3 (b) show a front view in a state where a substrate Is of the condition determining apparatus 1 is mounted to the ferroconcrete (object) 2 (FIG. 3 (a)) and a plan view of the substrate 1s of the condition determining apparatus 1 (FIG. 3 (b)).

Referring to FIG. 3 (a), the substrate 1s of the condition determining apparatus 1 is mounted to a surface (e.g. bottom surface) of the ferroconcrete 2. Note here that the excitation section 1a and the magnetic measuring sections 1b are arranged on the substrate 1s (see FIG. 3 (b)). It is also noted that the excitation signal generating section 1c, the correspondence recording section 1d, and the condition determining section 1e are arranged at positions apart from the substrate 1s.

Referring to FIG. 3 (b), the excitation section 1a is, for example, an elliptical excitation coil. Note here that the excitation coil is not limited to have such an elliptical shape, but may be circular, quadrilateral, or linear. Also referring to FIG. 3 (b), the excitation section 1a is a single excitation coil, but there may be multiple excitation coils. In this case, the multiple excitation coils may or may not have the same excitation frequency.

The magnetic measuring sections 1b are each arranged to measure a magnetic field generated by an eddy current EC that is generated on the rebar (measuring target) 2a. The multiple magnetic measuring sections 1b are provided. The magnetic measuring sections 1b are each arranged to perform Fourier transform or detection with an excitation signal to output the amplitude and phase of a signal based on the magnetic field as a measurement result. Note here that the amplitude and phase is merely an example measurement result and the following t-value may be another measurement result.

$$t = \frac{\vert {x}_{1} - {x}_{2} \vert}{\sqrt{\frac{{s}_{1}^{2}}{n} + \frac{{s}_{2}^{2}}{n}}}$$

where x₁ represents the average of the phases of the normal rebar 2a, x₂ represents the average of the phases of a rebar 2 with corrosion 2c, s₁² represents the phase unbiased variance of the normal rebar 2a, s₂² represents the phase unbiased variance of the rebar 2 with the corrosion 2c, and “n” represents the number of measurements. Since the numerator of the t-value represents the phase difference between the normal and corroded rebars 2a and the denominator represents the variance, the higher the t-value, the greater the difference between the measurement results of the normal and corroded rebars 2a. It is noted that the t-value can also be calculated in amplitude, though calculated in phase above.

In the embodiment of the present invention, the magnetic measuring sections 1b are magnetic sensors. The magnetic measuring sections (magnetic sensors) 1b are each arranged to measure a magnetic field in, for example, three axes (X, Y, and Z axes) (see FIGS. 3 (a) and 3 (b)), but may be arranged to measure a magnetic field in two axes or in one axis. It is noted that the magnetic measuring sections 1b may not be magnetic sensors, but may be coils for magnetic measurement. Also, the X-axis direction corresponds to the longitudinal direction of the rebar 2a, the Z-axis direction is perpendicular to the surface of the sheet in FIG. 3 (b), and the Y-axis direction is perpendicular to the X-axis and the Z-axis.

Referring to FIG. 3 (b), the multiple magnetic measuring sections (magnetic sensors) 1b are arranged on the substrate 1s at equal intervals in each column (Y-axis direction) and also in each row (X-axis direction). The excitation section (excitation coil) 1a is arranged immediately above the central magnetic measuring section 1b. It is noted that the arrangement may not be at equal intervals, though described above “at equal intervals” in each column and row. The magnetic measuring sections 1b may also be arranged linearly (e.g. in the X-axis or Y-axis direction) or three-dimensionally (e.g. in the X-axis, Y-axis, and Z-axis directions). Alternatively, the magnetic measuring sections 1b may be arranged concentrically.

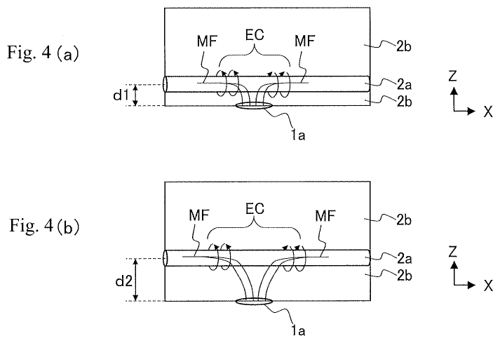

FIGS. 4 (a) and 4 (b) show an eddy current EC that is generated by the magnetic fields MF from the excitation section 1a in the cases where the normal rebar 2a is placed shallower (at a depth d1) (FIG. 4 (a)) and the normal rebar 2a is placed deeper (at a depth d2) (FIG. 4 (b)). Note here that in FIGS. 4 (a) and 4 (b), the rebar 2a is assumed to be normal (i.e. neither corroded nor broken).

Referring to FIGS. 4 (a) and 4 (b), the magnetic fields MF from the excitation section 1a are applied to the rebar 2a. The magnetic fields MF cause the eddy current EC to be generated on the rebar 2a. The magnetic measuring sections 1b are each arranged to measure a magnetic field generated by the eddy current EC.

As shown in FIG. 4 (a), in the case where the rebar 2a is placed shallower (at the depth d1), the magnetic field lines of the magnetic fields MF from the excitation section 1a rise approximately vertically (in the Z-axis direction) from the excitation section 1a into the rebar 2a with less spreading in the X-axis direction and then extend in the X-axis direction. The eddy current EC thus has an X coordinate close to that of the excitation section 1a. Accordingly, the magnetic field generated by the eddy current EC is also measured mostly by some of the magnetic measuring sections 1b close to the excitation section 1a.

As shown in FIG. 4 (b), in the case where the rebar 2a is placed deeper (at the depth d2), the magnetic field lines of the magnetic fields MF from the excitation section 1a spread widely in the X-axis direction into the rebar 2a and then extend in the X-axis direction. The eddy current EC thus has an X coordinate away from that of the excitation section 1a. Accordingly, the magnetic field generated by the eddy current EC is also measured mostly by some of the magnetic measuring sections 1b away from the excitation section 1a.

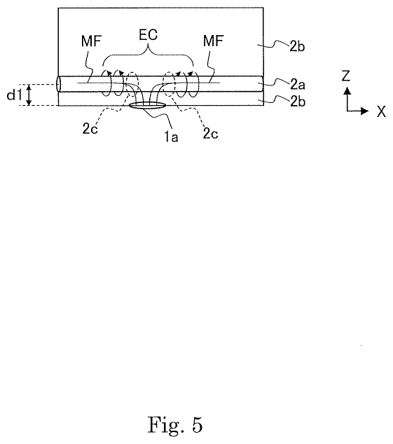

FIG. 5 shows an eddy current EC that is generated by the magnetic fields MF from the excitation section 1a in the cases where the rebar 2a with corrosion 2c is placed shallower (at the depth d1).

The rebar 2a with the corrosion 2c has a conductivity lower at the corrosion 2c than a normal portion of the rebar 2a. Also, the corrosion 2c generally has a specific permeability lower than that of the normal portion of the rebar 2a, though depending on corrosion products generated in the corrosion 2c. Accordingly, when the magnetic fields MF are applied from the excitation section 1a into the rebar 2a, the magnitude of the eddy current EC generated on the surface of the corrosion 2c is lower than the magnitude of the eddy current EC generated on the surface of the normal portion of the rebar 2a. Thus, the magnetic field on the surface of the corrosion 2c generated by the eddy current EC is measured by the magnetic measuring sections 1b to result in a lowered magnetic field amplitude and a smaller phase lag compared to those of the normal portion.

Here, as shown in FIG. 5, if the corrosion 2c has an X coordinate close to that of the excitation section 1a, the eddy current EC has a lower magnitude on the surface of the corrosion 2c, while has a higher magnitude in a portion away from the X coordinate of the excitation section 1a. That is, as shown in FIG. 5, if the corrosion 2c has an X coordinate close to that of the excitation section 1a, the magnetic field generated by the eddy current EC is measured mostly by some of the magnetic measuring sections 1b away from the excitation section 1a, even though the rebar 2a is placed shallower (at the depth d1).

Hence, even if the magnetic field generated by the eddy current EC may be measured mostly by some of the magnetic measuring sections 1b away from the excitation section 1a, it is not necessarily the case that the rebar 2a is placed deeper (FIG. 4 (b)) (the rebar 2a is normal), but the rebar 2a may be placed shallower (FIG. 5) (the rebar 2a may have corrosion 2c).

It is noted that the same applies even if the corrosion 2c may not be corroded but broken.

The excitation signal generating section 1c is arranged to provide an excitation signal (e.g. an electrical signal) to the excitation section 1a.

The correspondence recording section 1d is arranged to record the correspondence between the condition of the measuring target (rebar 2a) as well as the depth of the measuring target (rebar 2a) and data based on measurement results from the magnetic measuring sections 1b. Note here that the data includes measurement results from the magnetic measuring sections 1b. Also, the recorded contents in the correspondence recording section 1d are recorded when the measuring target is placed at multiple respective depths in the object (ferroconcrete 2). Further, the condition of the measuring target is whether or not the rebar 2a is corroded (or broken).

For example, the correspondence recording section 1d is arranged to record measurement results (e.g. amplitudes and phases) from the magnetic measuring sections 1b when the rebar 2a is not corroded (i.e. normal) and placed at respective depths d1 and d2 (see FIGS. 4 (a) and 4 (b)). It is noted that the correspondence recording section 1d may be arranged to record measurement results from the magnetic measuring sections 1b for three or more depths of the rebar 2a, not limited to the two depths d1 and d2.

The condition determining section 1e is arranged to determine the condition of the measuring target based on measurement results from the magnetic measuring sections 1b, recorded contents in the correspondence recording section 1d, and the depth of the measuring target (rebar 2a), which is known.

It is noted that the condition determining section 1e may be arranged to determine the condition of the rebar 2a based on some of the measurement results from the magnetic measuring sections 1b. For example, the condition of the rebar 2a may be determined based only on measurement results from the magnetic measuring sections 1b in one row (X direction) immediately below the rebar 2a.

It is noted that the condition determining section 1e may be arranged to measure the position (e.g. XY coordinates) of the rebar 2a based on the measurement results from the magnetic measuring sections 1b. When the magnetic measuring sections 1b are positioned immediately below the rebar 2a, the magnetic field generated by the eddy current EC is increased, so that the position of the rebar 2a can be measured.

The condition determining section 1e may also be arranged to measure the diameter or radius of the rebar 2a based on the measurement results from the magnetic measuring sections 1b. The smaller the diameter of the rebar 2, the lower the magnitude of the eddy current EC generated on the surface of the rebar 2, so that the diameter or radius of the rebar 2a can be measured.

Next will be described an operation according to the first embodiment.

First, the substrate 1s of the condition determining apparatus 1 is mounted to the bottom surface of the ferroconcrete 2 (see FIG. 3 (a)). When the excitation signal generating section 1c provides an excitation signal to the excitation section 1a, the rebar 2a is excited by the excitation section 1a (to generate a magnetic field MF) and thereby an eddy current EC is generated on the surface of the rebar 2a.

Further, the substrate 1s of the condition determining apparatus 1 (see FIG. 3 (b)) is scanned in the X-axis direction and the Y-axis direction and arranged such that the magnetic field measured by the magnetic measuring sections 1b (i.e. the magnetic field generated by the eddy current EC that is generated on the rebar 2a) has an approximately maximum amplitude (i.e. the substrate 1s of the condition determining apparatus 1 is arranged immediately below the rebar 2a).

It is here assumed that the correspondence recording section 1d records, for example, measurement results (e.g. amplitudes) from the magnetic measuring sections 1b when the normal rebar 2a is placed at respective depths d1 and d2 (see FIGS. 4 (a) and 4 (b)). It is further assumed that the measurement results from the magnetic measuring sections 1b correspond to when the normal rebar 2a is placed at the depth d2 (the eddy current EC has an X coordinate away from that of the excitation section 1a). That is, it is assumed that the magnetic field generated by the eddy current EC is measured mostly by some of the magnetic measuring sections 1b away from the excitation section 1a.

In such a case, if it is known that the rebar 2a is placed at the depth d2, the condition determining section 1e determines that the rebar 2a is normal (see FIG. 4 (b)). On the other hand if it is known that the rebar 2a is placed at the depth d1, the condition determining section 1e determines that the rebar 2a is corroded (see FIG. 5).

In accordance with the first embodiment, the recorded contents in the correspondence recording section 1d are recorded when the rebar 2a is placed at the multiple respective depths d1 and d2 in the ferroconcrete 2 (see FIGS. 4 (a) and 4 (b)). Accordingly, when the condition (the presence of corrosion or breakage) of the rebar 2a inside the ferroconcrete 2 is determined based on the eddy current EC that is generated by exciting the rebar 2a, the difference in the eddy current EC at the depths d1 and d2 of the rebar 2a in the ferroconcrete 2 can be accommodated (see FIGS. 4 (a) and 4 (b)).

### Second Embodiment

The condition determining apparatus 1 according to a second embodiment is different from that of the first embodiment in that the data based on the measurement results from the magnetic measuring sections 1b is obtained through multivariate analysis of the measurement results from the magnetic measuring sections 1b.

The condition determining apparatus 1 according to the second embodiment includes an excitation section 1a, magnetic measuring sections 1b, an excitation signal generating section 1c, a correspondence recording section 1d, and a condition determining section 1e. Components identical to those in the first embodiment will hereinafter be designated by the same reference numerals to omit the descriptions thereof.

The excitation section 1a, the magnetic measuring sections 1b, and the excitation signal generating section 1c are the same as those in the first embodiment and will not be described.

The correspondence recording section 1d is arranged to record the correspondence between the condition of the measuring target (rebar 2a) and data based on measurement results from the magnetic measuring sections 1b. Note here that the data is obtained through multivariate analysis (e.g. principal component analysis) of the measurement results from the magnetic measuring sections 1b. The other aspects of the correspondence recording section 1d are the same as those in the first embodiment and will not be described.

The condition determining section 1e is the same as that in the first embodiment. A specific example of determination by the condition determining section 1e will be described below in the case where the correspondence between data and the condition of the rebar 2a is recorded in the correspondence recording section 1d, the data obtained through principal component analysis of the measurement results from the magnetic measuring sections 1b.

Principal component analysis is also used in image compression, and such principal component analysis in image compression is applied to the embodiment of the present invention. The channel (CH) mapping of the amplitude and phase of signals based on magnetic fields as measured by the magnetic measuring sections 1b can be considered a kind of image. An approach for image compression can therefore be applied directly to the measurement results from the magnetic measuring sections 1b.

An example case will be described in which principal component analysis is applied to the amplitudes of signals based on magnetic fields in the X, Y, and Z-axis directions. A matrix D including amplitudes in the X, Y, and Z-axis directions is defined as in the following formula (1).

$$D = ( \begin{matrix}{A}_{x} \\ {A}_{y} \\ {A}_{z}\end{matrix} ) \qquad ( 1 )$$

where Ax, Ay, and Az represent, respectively, the amplitudes in the X, Y, and Z directions standardized to have a mean value of 0 and a standard deviation of 1, with a size of 1×nm. Note here that “n” represents the number of the magnetic measuring sections 1b in the Y-axis direction, while “m” represents the number of the magnetic measuring sections 1b in the X-axis direction. A matrix D′ is here defined by subtracting the mean value of the components in the q-th column of D from each component in the q-th column of D so that D has a mean value of 0 in the column direction (where q is an integer of any value equal to or higher than 1 and equal to or lower than nm). Eigenvalues λ and eigenvectors w are then calculated for the variance-covariance matrix D′^{T}D′ of D′. From the first and second eigenvectors w1 and w2, the first and second principal components p1 and p2 are then obtained by the following formulae (2) and (3).

$${p}_{1} = {D}^{′} {w}_{1} \qquad ( 2 )$$

$${p}_{2} = {D}^{′} {w}_{2} \qquad ( 3 )$$

A lot of information (in the number n×m) is thus compressed into the two first and second principal components. It is noted that the principal component analysis may be performed on the phases in the three axial directions or on data obtained by combining the amplitudes and the phases, though have been performed only on the amplitudes in the three axial directions.

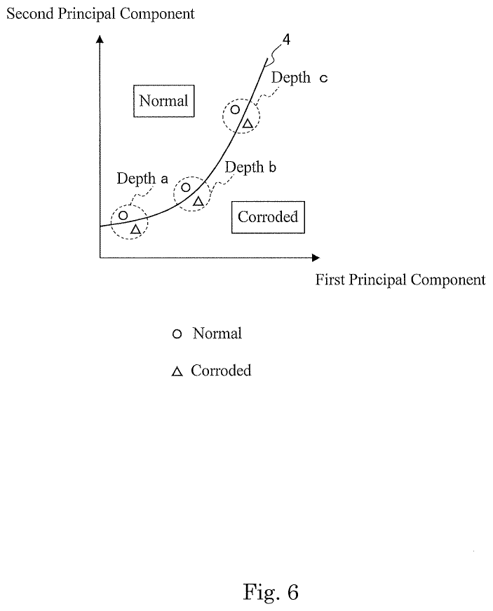

FIG. 6 illustrates determination of the condition of a measuring target (rebar 2a) by the condition determining section 1e according to the second embodiment.

First of all, for the case where the rebar 2a is normal and the case where the rebar 2a is corroded, the first and second principal components when the rebar 2a is placed at depths of a, b, and c (e.g. a=20 mm, b=40 mm, c=60 mm, though not particularly limited thereto) are plotted on a graph in which the horizontal axis represents the first principal component and the vertical axis represents the second principal component. Based on the plot, a boundary line 4 is generated by a support vector machine. The area above the boundary line 4 is determined to be normal, while the area below the boundary line 4 is determined to be corroded.

Next will be described an operation according to the second embodiment.

First, the substrate 1s of the condition determining apparatus 1 is mounted to the bottom surface of the ferroconcrete 2 (see FIG. 3 (a)). When the excitation signal generating section 1c provides an excitation signal to the excitation section 1a, the rebar 2a is excited by the excitation section 1a (to generate a magnetic field MF) and thereby an eddy current EC is generated on the surface of the rebar 2a.

Further, the substrate 1s of the condition determining apparatus 1 (see FIG. 3 (b)) is scanned in the X-axis direction and the Y-axis direction and arranged such that the magnetic field measured by the magnetic measuring sections 1b (i.e. the magnetic field generated by the eddy current EC that is generated on the rebar 2a) has an approximately maximum amplitude (i.e. the substrate 1s of the condition determining apparatus 1 is arranged immediately below the rebar 2a).

Also, the first and second principal components when the rebar 2a is placed at depths of a, b, and c have been recorded in the correspondence recording section 1d for the case where the rebar 2a is normal and the case where the rebar 2a is corroded (see FIG. 6). Note here that the correspondence recording section 1d is not required to record the depths of a, b, and c.

The condition determining section 1e here generates a boundary line 4 (see FIG. 6) from the recorded contents in the correspondence recording section 1d.

The condition determining section 1e further receives measurement results from the magnetic measuring sections 1b to obtain the first and second principal components. It is then determined whether the rebar 2a is normal or corroded based on whether the first and second principal components, when plotted on the graph in FIG. 6, are above or below the boundary line 4.

In accordance with the second embodiment, the recorded contents in the correspondence recording section 1d are recorded when the rebar 2a is placed at the multiple respective depths of a, b, and c in the ferroconcrete 2 (see FIG. 6). Accordingly, when the condition (the presence of corrosion or breakage) of the rebar 2a inside the ferroconcrete 2 is determined based on the eddy current EC that is generated by exciting the rebar 2a, the difference in the eddy current EC at the depths of the rebar 2a in the ferroconcrete 2 can be accommodated.

Moreover, in accordance with the second embodiment, the condition determining section 1e is arranged to determine whether the rebar 2a is normal or corroded based on whether the first and second principal components of the measurement results from the magnetic measuring sections 1b, when plotted, are above or below the boundary line 4 (see FIG. 6), whereby it is not particularly required that the depth of the rebar 2a is known.

It is noted that the second embodiment can include the following variation.

### VARIATION

In a variation of the second embodiment, the correspondence recording section 1d is arranged to record the correspondence between the condition and depth of the rebar 2a and data based on measurement results from the magnetic measuring sections 1b (through multivariate analysis of measurement results from the magnetic measuring sections 1b).

For example, the first and second principal components when the rebar 2a is placed at depths of a, b, and c have been recorded in the correspondence recording section 1d for the case where the rebar 2a is normal and the case where the rebar 2a is corroded (see FIG. 6). In addition, the correspondence recording section 1d has recorded the depths of a, b, and c.

The condition determining section 1e is arranged to determine the condition of the rebar 2a as is the case in the second embodiment. The condition determining section 1e is further arranged to measure the depth of the rebar 2a. For example, the condition determining section 1e is arranged to measure the depth of the rebar 2a based on which one of plots of the depths of a, b, and c the first and second principal components of the measurement results from the magnetic measuring sections 1b, when plotted on the graph in FIG. 6, are closest to. For example, if the first and second principal components, when plotted on the graph in FIG. 6, are closest to the plot of the depth of a, the rebar 2a is measured to have the depth of a.

### Third Embodiment

The condition determining apparatus 1 according to a third embodiment is different from that of the first embodiment in that the correspondence recorded in the correspondence recording section 1d is obtained through machine learning.

The condition determining apparatus 1 according to the third embodiment includes an excitation section 1a, magnetic measuring sections 1b, an excitation signal generating section 1c, a correspondence recording section 1d, and a condition determining section 1e. Components identical to those in the first embodiment will hereinafter be designated by the same reference numerals to omit the descriptions thereof.

The excitation section 1a, the magnetic measuring sections 1b, and the excitation signal generating section 1c are the same as those in the first embodiment and will not be described.

The correspondence recording section 1d is arranged to record the correspondence between the condition of the measuring target (rebar 2a) and data based on measurement results from the magnetic measuring sections 1b (e.g. the measurement results from the magnetic measuring sections 1b). Note here that the correspondence is obtained through machine learning with the condition of the rebar 2a and the measurement results from the magnetic measuring sections 1b as training data. It is also noted the recorded contents in the correspondence recording section 1d are recorded when the rebar 2a is placed at multiple respective depths in the ferroconcrete 2, as is the case in the first embodiment.

A method for such machine learning can employ one of convolutional neural networks (CNN), neural networks, and other well-known machine learning methods.

For example, when a convolutional neural network (CNN) is used as a method for machine learning, the determination model includes an input layer, a convolutional layer, a pooling layer, a fully connected layer, and an output layer. Then, n×m×6 data are input to the input layer. Here, 6 means the amplitude and phase for three axes. Note here that not all but only part of the phase and amplitude for the X, Y, and Z axes may be input to the input layer.

When the measurement results from the magnetic measuring sections 1b are provided to the input layer, an output from the input layer is provided to the convolutional layer, an output from the convolutional layer is provided to the pooling layer, an output from the pooling layer is provided to the fully connected layer, and an output from the fully connected layer is provided to the output layer. The condition (whether normal or corroded) of the measuring target (rebar 2a) is then output from the output layer.

Also, a sigmoid function is used as the activation function for the output layer. Note here that a Softmax function may alternatively be used as the activation function for the output layer and, in this case, the degree of corrosion (e.g. high corrosion, medium corrosion, and low corrosion) can be output as a multi-level output.

The condition determining section 1e is arranged to determine the condition of the rebar 2a based on measurement results from the magnetic measuring sections 1b and recorded contents in the correspondence recording section 1d.

Next will be described an operation according to the third embodiment.

First, the substrate 1s of the condition determining apparatus 1 is mounted to the bottom surface of the ferroconcrete 2 (see FIG. 3 (a)). When the excitation signal generating section 1c provides an excitation signal to the excitation section 1a, the rebar 2a is excited by the excitation section 1a (to generate a magnetic field MF) and thereby an eddy current EC is generated on the surface of the rebar 2a.

Further, the substrate 1s of the condition determining apparatus 1 (see FIG. 3 (b)) is scanned in the X-axis direction and the Y-axis direction and arranged such that the magnetic field measured by the magnetic measuring sections 1b (i.e. the magnetic field generated by the eddy current EC that is generated on the rebar 2a) has an approximately maximum amplitude (i.e. the substrate 1s of the condition determining apparatus 1 is arranged immediately below the rebar 2a).

The condition determining section 1e receives measurement results from the magnetic measuring sections 1b. The condition determining section 1e further retrieves the condition of the rebar 2a according to the measurement results from the magnetic measuring sections 1b from the recorded contents in the correspondence recording section 1d.

In accordance with the third embodiment, the recorded contents in the correspondence recording section 1d are recorded when the rebar 2a is placed at the multiple respective depths in the ferroconcrete 2. Accordingly, when the condition (the presence of corrosion or breakage) of the rebar 2a inside the ferroconcrete 2 is determined based on the eddy current EC that is generated by exciting the rebar 2a, the difference in the eddy current EC at the depths of the rebar 2a in the ferroconcrete 2 can be accommodated.

Moreover, in accordance with the third embodiment, it is not particularly required that the depth of the rebar 2a is known.

It is noted that the third embodiment can include the following variation.

### First Variation

It is noted that in a first variation of the third embodiment, the correspondence that the correspondence recording section 1d records is obtained through machine learning with the condition of the rebar 2a, the depth of the rebar 2a, and the measurement results from the magnetic measuring sections 1b as training data. This allows the condition determining section 1e to measure the depth of the rebar 2a.

### Second Variation

It is noted that in a second variation of the third embodiment, the correspondence is different for each of different depths of the rebar 2a. For example, the correspondence is recorded differently for each of depths of the rebar 2a of 0 to 20 mm, 20 to 40 mm, and over 40 mm. This can result in an improvement in the accuracy of the correspondence that is obtained through machine learning.

The above-described embodiments may also be implemented as follows. A computer including a CPU, a hard disk, and a medium (USB memory, CD-ROM, or the like) reading device is caused to read a medium with a program recorded thereon that achieves the above-described components (e.g. the correspondence recording section 1d and the condition determining section 1e) and install the program in the hard disk. The above-described features can also be achieved in this manner.

### DESCRIPTION OF REFERENCE NUMERALS

1 Condition Determining Apparatus
1a Excitation section
1b Magnetic Measuring Section
1c Excitation signal Generating Section
1d Correspondence Recording Section
1e Condition Determining Section
1s Substrate
2 Ferroconcrete (Object)
2a Rebar (Measuring Targe)
2b Concrete
2c Corrosion
4 Boundary Line
MF Magnetic Fields
EC Eddy Current
d1, d2, a, b, c Depth

What is claimed is:

1. A condition determining apparatus, comprising:
an excitation section arranged to excite a measuring target inside an object;
a plurality of magnetic measuring sections arranged to measure a magnetic field generated by an eddy current that is generated on the measuring target;
a correspondence recording section arranged to record a correspondence between a condition of the measuring target and data based on measurement results from the magnetic measuring sections; and
a condition determining section arranged to determine the condition of the measuring target based on measurement results from the magnetic measuring sections and recorded contents in the correspondence recording section, wherein
the recorded contents in the correspondence recording section are recorded when the measuring target is placed at a plurality of respective depths in the object.

2. The condition determining apparatus according to claim 1, wherein
the correspondence recording section is arranged to record a correspondence between the condition of the measuring target as well as the depth of the measuring target and the data,
the condition determining section is arranged to determine the condition of the measuring target based on the depth of the measuring target, which is known, and
the data includes measurement results from the magnetic measuring sections.

3. The condition determining apparatus according to claim 1, wherein
the data is obtained through multivariate analysis of measurement results from the magnetic measuring sections.

4. The condition determining apparatus according to claim 1, wherein
the correspondence recording section is arranged to record a correspondence between the condition of the measuring target as well as the depth of the measuring target and the data,
the condition determining section is arranged to determine the condition of the measuring target and further to measure the depth of the measuring target, and
the data is obtained through multivariate analysis of measurement results from the magnetic measuring sections.

5. The condition determining apparatus according to claim 1, wherein
the correspondence is obtained through machine learning with the condition of the measuring target and the measurement results from the magnetic measuring sections as training data.

6. The condition determining apparatus according to claim 1, wherein
the correspondence is obtained through machine learning with the condition of the measuring target, the depth of the measuring target, and the measurement results from the magnetic measuring sections as training data.

7. The condition determining apparatus according to claim 6, wherein
the correspondence varies depending on the depth of the measuring target.

8. The condition determining apparatus according to claim 1, wherein
the object is a ferroconcrete, and
the measuring target is a rebar.

9. The condition determining apparatus according to claim 8, wherein
the condition is whether or not the rebar is corroded.

10. The condition determining apparatus according to claim 8, wherein
the condition is whether or not the rebar is broken.

11. The condition determining apparatus according to claim 8, wherein
a position of the rebar is measured based on the measurement results from the magnetic measuring sections.

12. The condition determining apparatus according to claim 8, wherein
a diameter or radius of the rebar is measured based on the measurement results from the magnetic measuring sections.

13. The condition determining apparatus according to claim 1, wherein
the condition determining section is arranged to determine the condition of the measuring target based on some of the measurement results from the magnetic measuring sections.

14. A condition determining method, comprising:
exciting a measuring target inside an object;
measuring a magnetic field generated by an eddy current that is generated on the measuring target;
recording a correspondence between a condition of the measuring target and data based on measurement results from the measuring; and
determining the condition of the measuring target based on measurement results from the measuring and recorded contents in the recording, wherein
the recorded contents in the recording are recorded when the measuring target is placed at a plurality of respective depths in the object.

15. A non-transitory computer-readable medium including a program of instructions for execution by a computer to perform a condition determining process with using a condition determining apparatus including an excitation section arranged to excite a measuring target inside an object, and a plurality of magnetic measuring sections arranged to measure a magnetic field generated by an eddy current that is generated on the measuring target, the condition determining process comprising:
recording a correspondence between a condition of the measuring target and data based on measurement results from the magnetic measuring sections; and
determining the condition of the measuring target based on measurement results from the magnetic measuring sections and recorded contents in the recording, wherein
the recorded contents in the recording are recorded when the measuring target is placed at a plurality of respective depths in the object.
